(19) United States
(12) Patent Application Publication
(10) Pub. No.: US 2026/0369393 A1
(43) Pub. Date: Nov. 19, 2026

(54) COMPUTER-IMPLEMENTED METHOD FOR DEFINING A PROCESS PARAMETER IN A MEDICAL FACILITY

(71) Applicant: DEO N.V., Beringen (BE)

(72) Inventors: Niels FESTJENS, Beringen (BE); Jeroen Willem Rita DILLE, Beringen (BE)

(73) Assignee: DEO N.V., Beringen (BE)

(21) Appl. No.: 19/487,911

(22) PCT Filed: Jun. 13, 2024
(86) PCT No.: PCT/EP2024/066468

§ 371 Date: Nov. 25, 2025

(30) Foreign Application Priority Data
Jun. 16, 2023 (NL) 2035113

(51) Int. Cl. G16H 40/20

(52) U.S. Cl. CPC G16H 40/20

(57) ABSTRACT

Computer-implemented method for evaluating at least one process parameter of a process in a medical room, the method comprising the steps of: receiving medical room sensor data monitored in said medical room; determining, from said received medical room sensor data, at least one process parameter which is indicative for the operational performance of the process in said medical room; associating at least one process indicator with the determined process parameter, wherein a process indicator is indicative for a process type in said medical room; providing at least one historical process parameter associated with the process indicator; normalizing said determined process parameter on the basis of the provided at least one historical process parameter; and outputting the normalized process parameter.

### FIELD OF INVENTION

The field of the invention relates to a computer-implemented method for defining a process parameter in a medical facility.

### BACKGROUND

Healthcare or medical facilities need to address an increasing demand for timely and high-quality care for their patients whilst overcoming shortages in medical staff and other challenges.

Healthcare services are however a complex system of operationally interlinked processes, For example, a substantial amount of processes are directed at resource allocation where, for example, the right tools for a surgeon need to be in the right room at the right time. Another example is the administrative load where patient scheduling has a direct impact on the organization of an operating room (OR) and the medical facility in general. Scheduling is directly linked to the planning of human, material and infrastructure resources. It also immediately impacts short term impact on OR throughput. Whilst the above challenges are situated at a relatively high or, put differently, macro level, the same, as well as other problems exist at a micro level. The micro level of a hospital or medical facility is typically a medical room such as an operating room. Also at a micro level, the operational challenges are substantial. For example, an erroneous posture of a nurse throughout a medical intervention can lead to physical hurt and drop-out of the nurse for recovering from the nurse is to be expected. It has been shown that all of these challenges have a direct impact on the recovery of patients in the medical facility, notwithstanding a direct impact on the health of the medical staff.

In order to address these challenges enhancing efficiency, optimizing medical patterns and upskilling of healthcare professionals is required. However, in order to improve the processes, the current state of the processes has to be observed prior to improving the processes. Based on the observations suggestions may be made based on one or more parameters. However, the shear quantity of processes, the different kinds of processes, the specificities of the processes as well as the skills of the observer which are required to perform the observations of the parameters prove to be a challenge which currently has yet to be overcome.

### SUMMARY

The object of embodiments of the present invention is to provide a computer-implemented method for defining at least one process parameter being indicative of an operational performance.

According to a first aspect of the present invention there is provided a computer-implemented method for defining at least one process parameter being indicative of an operational performance of a process in a medical facility. The method comprises the following steps of:
receiving medical room sensor data monitored in said at least one medical room of said medical facility;
determining, from said received medical room sensor data, at least one process parameter which is indicative for the operational performance of the process in said medical room;
associating at least one process indicator with the determined process parameter, wherein a process indicator is indicative for a process type in said medical room;
providing at least one historical process parameter associated with the process indicator;
normalizing said determined process parameter on the basis of the provided at least one historical process parameter;
outputting the normalized process parameter.

The advantage of the method is based on the insight that by implementing the computer-implemented method, medical facilities can effectively monitor and evaluate the operational performance of processes within their medical rooms. The method enables the identification of process parameters, association with process indicators, historical benchmarking, normalization, and outputting of normalized process parameters for improved process management and performance optimization. The computer-implemented method initially receives sensor data from at least one medical room in the medical facility. Based on the received sensor data, the method determines at least one process parameter that is indicative of the operational performance of the process in the medical room. The process parameters could include metrics such as room temperature stability, air quality, equipment performance, a distance walked, tools pick up and placed back down, length of conversations etc. or any other factors that impact the process within the medical room. The determined process parameter is associated with at least one process indicator. A process indicator represents a specific process type taking place in the medical room. For example, the process type could be surgical procedures, patient monitoring, or laboratory tests. The association links the process parameter to the specific process type. Next, the method retrieves at least one historical process parameter associated with the process indicator. This historical data can represent previous measurements or benchmarks related to the same process type in the medical room. The historical data provides a reference point for evaluating the current operational performance. The determined process parameter is normalized based on the provided historical process parameter. Normalization ensures that the current measurement is compared and adjusted against the historical data to account for any variations or changes over time. This step allows for a standardized and comparable assessment of the operational performance. Finally, the method outputs the normalized process parameter, which represents the operational performance of the process in the medical room. This output can be utilized for analysis, visualization, reporting, or further decision-making processes related to optimizing the performance of the process.

Preferably, the historical process data containing the process indicator which indicative for the same process type comprises previously determined process parameters associated with the process indicator indicative for the same process type and/or predetermined process parameters associated with the process indicator indicative for the same process type, wherein the predetermined process parameters represent best practice process parameters for said process type. In this way, the computer-implemented method incorporates historical process data that contains the process indicator indicative of the same process type. This historical process data includes two types of process parameters or a combination thereof. Firstly, previously determined process parameter parameters are associated with the process indicator indicative of the same process type. The previously determined process parameters represent measurements or benchmarks that have been previously determined or recorded for similar processes in the medical room, for example an knee replacement performed by the same medical team. This data provides a historical reference to assess the current operational performance. Alternatively or additionally, the historical process data can also include predetermined process parameters associated with the process indicator indicative of the same process type. These predetermined process parameters represent the best practice or ideal values for the specific process type in the medical room. They serve as a standard or target to evaluate and optimize the operational performance. By incorporating both previously determined process parameters and predetermined process parameters, the method enables a comprehensive evaluation of the current operational performance. The historical data provides insights into past performance, while the predetermined process parameters represent an optimal or desired state for the process type. This combination allows for a more robust assessment and facilitates the identification of areas for improvement or adherence to best practices.

Preferably, the computer-implemented method further comprises determining the process indicator from the medical room sensor data. This step allows for the automated identification of the specific process type taking place in the medical room based on the sensor data. The determination of the process indicator can be achieved through various techniques, such as pattern recognition, machine learning algorithms, or rule-based systems. By determining the process indicator, the method gains the ability to associate the appropriate process parameters and historical data specific to that process type. This ensures that the analysis and evaluation of the operational performance are tailored to the particular process being conducted in the medical room. It also allows for the utilization of process-specific best practice parameters for optimization purposes. Including the determination of the process indicator enhances the accuracy, relevance, and effectiveness of the method in assessing and improving the operational performance of processes in the medical facility. By leveraging the medical room sensor data to automatically identify the process type, the method can provide more targeted insights and recommendations for process optimization, ultimately leading to enhanced operational efficiency and quality of care.

Preferably, the computer-implemented method further comprises receiving medical room process data containing at least one process indicator which is indicative for a process type in said medical room. More preferably, the computer-implemented method further comprises the steps of including the determined process parameter associated with the process indicator in the historical process data. In the computer-implemented method, it is preferable to include the step of receiving medical room process data containing at least one process indicator that is indicative of a process type in the medical room. This additional step allows for the incorporation of process-specific information and measurements into the analysis and evaluation of the operational performance. Furthermore, it is more preferable to include the step of including the determined process parameter associated with the process indicator in the historical process data. This ensures that the newly determined process parameter is added to the existing historical data for that particular process type. By including the determined process parameter, the historical process data becomes more comprehensive and up-to-date, facilitating a more accurate assessment and comparison of the operational performance over time. By receiving medical room process data and including the determined process parameter in the historical process data, the computer-implemented method enables a continuous and dynamic monitoring and evaluation of the operational performance of processes in the medical room. The method can adapt to changes and updates in the process parameters, ensuring that the assessment remains relevant and reflective of the current state of the process. This improved approach allows for a more informed analysis, benchmarking, and optimization of the operational performance.

Preferably, said medical room sensor data comprises video data of the medical room, and/or audio data obtained in the medical room. More preferably, said medical room sensor data comprises multi-source medical room sensor data, wherein at least two of the sources comprises a respective intake angle of the medical room. In the computer-implemented method, it is preferable to include video data and/or audio data of the medical room as part of the medical room sensor data. Video data provides visual information about the medical room, including the layout, equipment, and activities taking place. Audio data captures sound information, which can be valuable for assessing noise levels, communication quality, or the presence of specific sounds relevant to the process being evaluated. Additionally, it is more preferable to include multi-source medical room sensor data, where at least two of the sources provide a respective intake angle of the medical room. The intake angle refers to the perspective or viewpoint from which the medical room is observed or captured. By incorporating multiple intake angles, the method gains a more comprehensive and nuanced understanding of the medical room's environment, activities, and process performance. By including video data, audio data, and multi-source sensor data with various intake angles, the computer-implemented method can leverage a rich and diverse set of information for determining process parameters and assessing operational performance. The combination of visual and audio cues, along with multiple perspectives, provides a more holistic view of the medical room and its processes. This approach enhances the accuracy and effectiveness of the method in analysing and evaluating the operational performance of processes. It allows for a deeper understanding of the context, interactions, and potential influencing factors within the medical room, contributing to more informed decision-making and optimization efforts.

Preferably, the process parameter is indicative for the ergonomic position of at least one person in the medical room. In the computer-implemented method, it is preferable for the process parameter to be indicative of the ergonomic position of at least one person in the medical room. Ergonomic position refers to the posture and positioning of a person in a way that promotes comfort, safety, and efficiency. Furthermore, it is more preferable for the method to include the step of estimating the pose of the at least one person in the medical room. Estimating the pose involves analysing the video data or sensor data to determine the spatial configuration and position of the person's body parts. This can be achieved using techniques such as pose estimation, skeleton tracking, or motion capture. By estimating the pose of the person, the method gains the ability to assess the ergonomic position and alignment of a body of a person in the medical room. The process parameter derived from this assessment indicates whether the person's posture is ergonomically optimal or whether there are potential issues that could lead to discomfort, fatigue, or injury. Including ergonomic considerations and pose estimation in the computer-implemented method allows for a more comprehensive evaluation of the operational performance in the medical room. It helps identify potential ergonomic risks, evaluate the impact of body positioning on efficiency and safety, and provide feedback or recommendations to improve the ergonomic conditions for the personnel involved in the processes. By integrating ergonomic considerations and pose estimation, the method contributes to creating a healthier and safer work environment in the medical facility, ultimately enhancing the well-being and productivity of the personnel while optimizing the overall operational performance.

More preferably, the computer-implemented method further comprises tracking of variations of the pose estimation between digital images comprised in the video data. This additional step allows for the analysis and monitoring of changes in the pose of the person over time, providing insights into their movement patterns and potential ergonomic issues. By tracking variations of the pose estimation, the method can detect and quantify any deviations or fluctuations in the person's posture and body position. This information can be used to identify repetitive or prolonged movements, postural instability, or other factors that may contribute to ergonomic challenges or potential musculoskeletal issues. The tracking of pose estimation variations adds a dynamic aspect to the evaluation of ergonomic positions in the medical room. It enables the method to capture not only static snapshots of the person's posture but also the changes and dynamics of their movements throughout the video data. This temporal analysis enhances the understanding of ergonomic risks and helps in identifying trends or patterns that may require intervention or adjustment. By incorporating the tracking of variations in pose estimation, the computer-implemented method provides a more comprehensive and detailed assessment of the ergonomic conditions and movement behaviours in the medical room. This information can be used to guide interventions, optimize workflow, and enhance the overall ergonomic design and practices in the medical facility.

Preferably, the step of determining the ergonomic process parameter comprises comparing the estimated pose to a baseline, and calculating at least one pose score based on the comparison. This step involves evaluating the alignment and positioning of the estimated pose in relation to a predetermined standard or reference pose, which represents the ergonomic baseline.

Furthermore, it is more preferable for the method to include the step of outputting the calculated pose score as the ergonomic process parameter. The pose score represents a quantitative measure of how closely the estimated pose aligns with the ergonomic baseline. It provides a numerical indication of the ergonomic quality of the person's posture and serves as a metric to assess their ergonomic performance.

Additionally, it is preferable to include the step of comparing the calculated pose score to historical process data that contains a process indicator indicative of the same process type in the medical room. By comparing the pose score to historical process data, the method establishes a context-specific evaluation of the ergonomic performance. This comparison allows for benchmarking the current pose score against past measurements or benchmarks associated with the same process type, enabling insights into the progress or deviations over time. By incorporating the steps of comparing the estimated pose to a baseline, calculating pose scores, outputting the pose scores as the ergonomic process parameter, and comparing the pose scores to historical process data, the computer-implemented method provides a comprehensive framework for assessing and monitoring ergonomic performance. This approach enables the identification of potential ergonomic issues, the evaluation of improvements or deteriorations, and the establishment of evidence-based guidelines for optimal ergonomic practices in the medical facility.

Preferably, the method further comprises outputting one or more pose proposals based on the comparison, wherein the one or more pose proposals represent at least part of a pose improvement to be carried out on a corresponding real body part of the at least one person in the medical room such that a difference between the calculated at least one pose score and the historical process data is reduced. These pose proposals represent suggested improvements to be made to the corresponding real body part of the person in the medical room. The aim of these proposals is to reduce the difference between the calculated pose score and the historical process data, indicating a closer alignment with optimal ergonomic conditions. It is noteworthy that the optimal ergonomic condition can be contextually determined based on the process type. For example, an upright ergonomic position of a person in the medical room may be the best-in-class without any process going on. However, when a surgical procedure is occurring, it is possible that the upright position is not possible due to the surgical steps needing to be performed. The optimal ergonomic position can therefore be process dependent.

Furthermore, it is more preferable to use RULA, Rapid Upper Limb Assessment, and/or REBA, Rapid Entire Body Assessment, scoring methods in the calculation of the pose scores. RULA and REBA are ergonomic assessment tools that provide structured methodologies for evaluating and scoring the ergonomic risks associated with body postures and movements. These scoring systems assign scores based on various factors such as posture, force exertion, repetition, and duration.

By incorporating RULA and/or REBA scoring in the calculation of the pose scores, the method applies recognized and validated ergonomic assessment methods. This allows for a standardized and objective evaluation of the ergonomic quality of the estimated pose. The pose proposals generated based on these scores provide actionable recommendations for improving the ergonomics of the person's posture and movements. The outputted pose proposals guide the person in making adjustments to their body position, posture, or movements to achieve a more optimal ergonomic alignment. By implementing these suggested improvements, the difference between the calculated pose score and the historical process data is expected to decrease, indicating progress towards better ergonomic performance. In summary, the inclusion of the step to output pose proposals, along with the use of RULA and/or REBA scoring, enhances the practical application of the computer-implemented method. It provides actionable recommendations for improving ergonomic conditions, promotes healthier and safer work practices, and facilitates the reduction of discrepancies between calculated pose scores and historical process data.

Preferably, the process parameter is indicative for a first difference between a first time stamp at which a first process type takes place and a second time stamp at which a second process type takes place, wherein the first process type corresponds to a first incision and the second process type corresponds to a wound closure of at least one person in the medical room. A time stamp refers to a piece of data that indicates the specific time at which an event occurred or a particular action took place. It is used to mark the moment when something happened, for example the first incision or the wound closure and provides a reference point in time for record-keeping, analysis, or synchronization purposes. An incision refers to a deliberate and controlled cut or opening made in the body, typically during a surgical procedure. It is a common medical term used to describe the act of making an intentional surgical cut through the skin, tissue, or organ to access a specific area for diagnosis, treatment, or other medical interventions. Incisions are performed by healthcare professionals, such as surgeons or medical practitioners, using specialized instruments like scalpels, surgical scissors, or lasers. When the incision is closed after a surgical procedure, it is commonly referred to as wound closure. Wound closure involves bringing the edges of the incision together and securing them to promote healing and minimize the risk of infection. A process parameter which represents the difference between the first time stamp, i.e. when the first incision is made, and the second time stamp, i.e. when the incision is closed, is indicative for the efficiency of the surgical process and has secondary effects on the treatment quality. For instance a shorter process parameter which represents the difference between the first time stamp and the second time stamp results in shorter sedation periods which leads to better recovery and reduction of other risks.

Preferably, the step of normalizing further comprises comparing the first difference to historical process data containing a process indicator which is indicative for the same process type. This normalization step allows for a contextual assessment of the change in the process parameter in relation to past measurements or benchmarks associated with the same process type. By comparing the first difference to historical process data, the method establishes a reference point to evaluate the significance and impact of the change in the process parameter. It provides a means to assess whether the observed change is within expected ranges or if it deviates from historical patterns. The comparison to historical process data allows for the identification of trends, anomalies, or potential issues in the operational performance of the process. It helps determine whether the change in the process parameter is consistent with past behaviour or if it requires further investigation or intervention. By incorporating this normalization step, the computer-implemented method ensures that the assessment of the process parameter takes into account the historical context and process-specific considerations. It provides a more accurate and meaningful evaluation of the operational performance, enabling timely identification of deviations and the implementation of appropriate measures to address them. In summary, the normalization step, which involves comparing the first difference to historical process data, enhances the method's ability to analyse and interpret the changes in the determined process parameter. It contributes to a more comprehensive understanding of the process performance and supports informed decision-making for process optimization and improvement. The difference can be associated with historical differences measured for example for the same team in the same setting, or in another setting, or another team.

Preferably, the historical process data containing the process indicator which is indicative for the same process type comprises a minimal historical difference between a first time stamp at which a first process type takes place and a second time stamp at which a second process type takes place, and a maximum historical difference between a first time stamp at which a first process type takes place and a second time stamp at which a second process type takes place, wherein the normalizing comprises performing a ration between a difference between the first difference and the minimal difference to a difference between the maximum difference and the minimum difference. The observed difference can be normalised by combining the difference with a large set of historical measurements in different settings, for instance by comparing to the shortest and longest documented difference of a group or a subgroup, e.g. all hospitals globally, all private clinics, all teams of the same medical department etc.

Preferably, the process parameter is indicative for at least one of an amount of verbally communicated words, a pace of oral communication, an amount of simultaneous oral communications, a time period between two oral communications. Put differently, it is preferable for the process parameter to be indicative of various aspects of oral communication. These aspects can include an amount of verbally communicated words such a process parameter can quantify the number of words spoken during a given period. It provides insights into the volume of information exchanged through oral communication. Another aspect of oral communication can be the pace of oral communication. Such a process parameter can measure the speed or rate at which oral communication occurs. It indicates the tempo or cadence of spoken interactions. Another aspect of oral communication can be the amount of simultaneous oral communications. This process parameter can assess the number of concurrent oral communications happening within a specific context or environment. It helps evaluate the level of multitasking or overlapping conversations. Yet another aspect of oral communication can be the time period between two oral communications. this process parameter can determine the duration between successive oral communications. It provides information about the intervals or gaps between spoken interactions. By utilizing these process parameters, the computer-implemented method can analyse and monitor various aspects of oral communication in a medical facility or any relevant context. These parameters offer insights into communication patterns, efficiency, and potential challenges within the oral communication process. The method can capture, measure, and analyse these process parameters using appropriate sensor data, such as audio recordings, speech recognition technologies, or other relevant sources. This enables a quantitative evaluation of oral communication performance and facilitates targeted improvements to enhance communication effectiveness, efficiency, and patient care in the healthcare facility. In summary, the inclusion of process parameters related to the amount of verbally communicated words, pace of oral communication, amount of simultaneous oral communications, and time period between two oral communications enhances the method's ability to evaluate and optimize oral communication practices in the medical facility or other relevant settings.

Preferably, the process parameter is indicative for a movement path for one or more objects in the medical room, and wherein the normalizing comprises comparing the movement path to historical process data containing a process indicator which is indicative for the same process type. More preferably, the normalizing further comprises overlaying the historical movement path data on to the determined movement path. This process parameter focuses on analysing the trajectory or path taken by objects within the medical room, providing insights into their movement patterns. To normalize the determined movement path, it is preferable to compare it to historical process data containing a process indicator indicative of the same process type. By doing so, the method establishes a reference point to evaluate the current movement path in relation to past measurements or benchmarks associated with similar processes. Furthermore, in the normalization step, it is more preferable to overlay the historical movement path data onto the determined movement path. This overlaying process allows for a direct visual comparison between the current movement path and the historical data. It helps identify similarities, differences, or deviations in the movement patterns of the objects within the medical room. By comparing and overlaying the movement paths, the method enables a comprehensive analysis of the current movement patterns in relation to historical data. This comparison helps identify any irregularities, variations, or areas of improvement in the movement path of objects within the medical room. The normalization step, including the comparison of movement paths and overlaying of historical data, provides a means to evaluate the efficiency, effectiveness, and safety of object movements in the medical room. It assists in identifying potential bottlenecks, areas of congestion, or suboptimal routes, leading to insights for process optimization and improvement. In summary, the incorporation of process parameters related to the movement path of objects in the medical room, along with the normalization step involving comparison and overlaying of historical movement path data, enhances the method's ability to analyse, compare, and optimize object movements. It enables a data-driven approach to improve the flow, efficiency, and safety of processes within the medical facility.

Preferably, the computer-implemented method further comprises indexing the video data and performing any one of the previously described steps for each indexed data point in the video data. This indexing process involves organizing and categorizing the video data into individual data points or segments for further analysis. Once the video data is indexed, the method can perform any of the previously described steps for each indexed data point. This means that the steps, such as receiving sensor data, determining process parameters, associating process indicators, providing historical process data, normalizing, and outputting results, are carried out for each segment of the video data. By applying the method to each indexed data point in the video data, the analysis becomes more granular and detailed. It allows for a more comprehensive evaluation of the operational performance, process indicators, and ergonomic factors within the medical room. The indexing process enables the method to analyse and assess the video data in a structured manner, segmenting it into manageable units for processing. This allows for targeted analysis of specific moments or events within the video data, facilitating accurate and meaningful evaluation of the process parameters and their variations over time. In summary, the inclusion of indexing in the computer-implemented method enhances the analysis and processing of video data in a systematic manner. It enables the application of the method's steps to each indexed data point, providing a detailed assessment of operational performance and process indicators in the medical room.

According to yet another aspect of the present invention, there is provided a computer program product comprising a computer-executable program of instructions for performing, when executed on a computer, the steps of the method of any one of the method embodiments described above.

It will be understood by the skilled person that the features and advantages disclosed hereinabove with respect to embodiments of the method may also apply, mutatis mutandis, to embodiments of the computer program product.

Further aspects of the present invention are described by the dependent claims. The features from the dependent claims, features of any of the independent claims and any features of other dependent claims may be combined as considered appropriate to the person of ordinary skill in the art, and not only in the particular combinations as defined by the claims.

### BRIEF DESCRIPTION OF THE FIGURES

The accompanying drawings are used to illustrate presently preferred non-limiting exemplary embodiments of devices of the present invention. The above and other advantages of the features and objects of the present invention will become more apparent and the present invention will be better understood from the following detailed description when read in conjunction with the accompanying drawings, in which:

FIG. 1 schematically shows an overview of the method according to an exemplary embodiment;

FIG. 2 schematically illustrates another embodiment of a according to the present invention, e.g. a further development of the embodiment shown in FIG. 1.

### DESCRIPTION OF EMBODIMENTS

A person of skill in the art would readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The program storage devices may be, e.g., digital memories, magnetic storage media such as a magnetic disks and magnetic tapes, hard drives, or optically readable digital data storage media. The program storage devices may be resident program storage devices or may be removable program storage devices, such as smart cards. The embodiments are also intended to cover computers programmed to perform said steps of the above-described methods.

The description and drawings merely illustrate the principles of the present invention. It will thus be appreciated that those skilled in the art will be able to devise various arrangements that, although not explicitly described or shown herein, embody the principles of the present invention and are included within its scope. Furthermore, all examples recited herein are principally intended expressly to be only for pedagogical purposes to aid the reader in understanding the principles of the present invention and the concepts contributed by the inventor(s) to furthering the art, and are to be construed as being without limitation to such specifically recited examples and conditions. Moreover, all statements herein reciting principles, aspects, and embodiments of the present invention, as well as specific examples thereof, are intended to encompass equivalents thereof.

The functions of the various elements shown in the figures, including any functional blocks labelled as “processors”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

It should be appreciated by those skilled in the art that any block diagrams herein represent conceptual views of illustrative circuitry embodying the principles of the present invention. Similarly, it will be appreciated that any flowcharts, flow diagrams, state transition diagrams, pseudo code, and the like represent various processes which may be substantially represented in computer readable medium and so executed by a computer.

It should be noted that the above-mentioned embodiments illustrate rather than limit the present invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim. The word “a” or “an” preceding an element does not exclude the presence of a plurality of such elements. The present invention can be implemented by means of hardware comprising several distinct elements and by means of a suitably programmed computer. In claims enumerating several means, several of these means can be embodied by one and the same item of hardware. The usage of the words “first”, “second”, “third”, etc. does not indicate any ordering or priority. These words are to be interpreted as names used for convenience.

In the present invention, expressions such as “comprise”, “include”, “have”, “may comprise”, “may include”, or “may have” indicate existence of corresponding features but do not exclude existence of additional features.

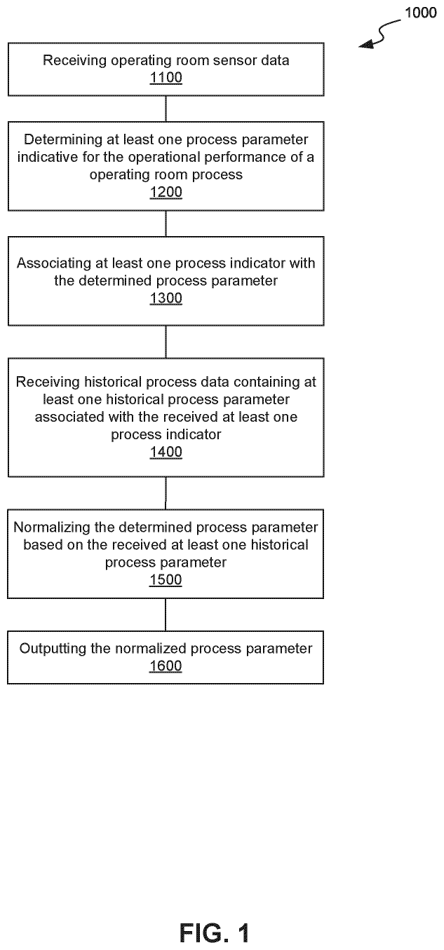

FIG. 1 illustrates a flowchart of an embodiment of a computer-implemented method 1000 for defining at least one process parameter being indicative of an operational performance of a process in a medical facility. In the context of the present application, a process parameter can be defined as a measurable or observable variable that is used to quantify or describe a specific aspect of a process in the medical facility. The at least one process parameter provides information or data about the state, performance, or characteristics of the process being analysed. Examples of a medical process are surgery which typically includes pre-operative assessments, patient preparation, administering anaesthesia, the actual surgical procedure, post-operative care, and monitoring. Surgery involves collaboration among surgeons, anaesthesiologists, nurses, and other supporting staff. Other examples of medical processes are infection control including regular hand hygiene practices, proper use of personal protective equipment, disinfection and sterilization of equipment and surfaces, and isolation precautions for patients with contagious conditions.

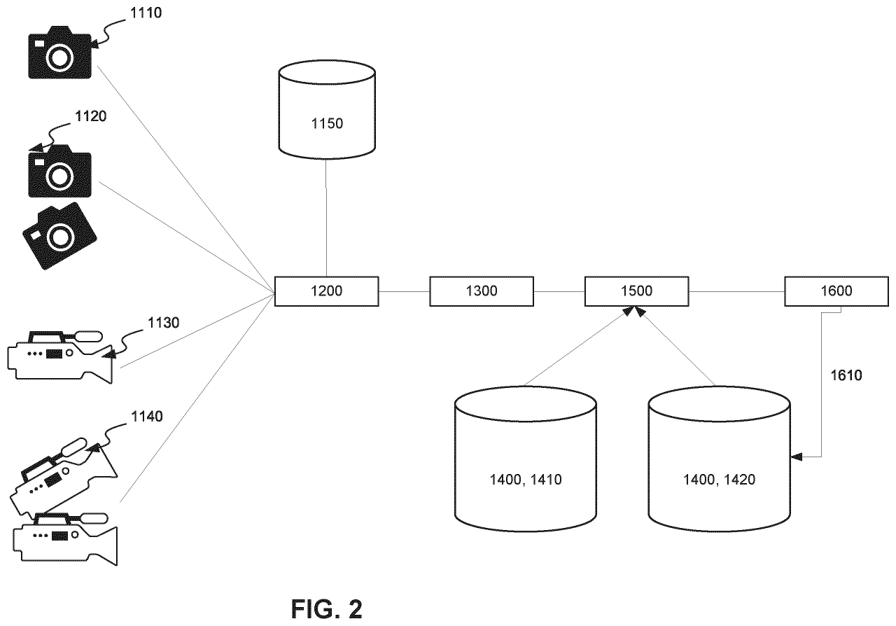

The computer-implemented method 1000 initially receives 1100 sensor data from at least one medical room in the medical facility. Examples of the sensor data are provided in relation to FIG. 2.

Based on the received 1100 sensor data, the method 1000 determines 1200 at least one process parameter that is indicative of the operational performance of the process in the medical room. By determining the process indicator, the method 1000 can associate the appropriate process parameters and historical data specific to that process type. This ensures that the analysis and evaluation of the operational performance are tailored to the particular process being conducted in the medical room. It also allows for the utilization of process-specific best practice parameters for optimization purposes. A process parameter can in this context be indicative for the ergonomic position of at least one person in the medical room, for a process type, for a first difference between a first time stamp at which a first process type takes place and a second time stamp at which a second process type takes place, for an amount of verbally communicated words, for a pace of oral communication, for an amount of simultaneous oral communications, for a time period between two oral communications, for a physical setup of an operating room, for sterility, for an environmental impact of the healthcare facility, for resource allocation, for physical or mental fatigue, for drug use, for synchronization etc. Further examples of process parameters could include metrics such as room temperature stability, air quality, equipment performance, a distance walked, tools pick up and placed back down, etc. or any other factors that impact the process within the medical room. Process parameters can be diverse and vary depending on the nature of the process and the specific goals of analysis. They can encompass a wide range of factors, including physical properties, operational variables, performance metrics, environmental conditions, or any other relevant aspects that contribute to understanding the process. The above mentioned example are non-limiting. Specific exemplary embodiments relating thereto will be elaborated here below. Preferably, the computer-implemented method 1000 further comprises determining the process indicator from the medical room sensor data. This step allows for the automated identification of the specific process type taking place in the medical room based on the sensor data. The determination of the process indicator can be achieved through various techniques, such as pattern recognition, machine learning algorithms, or rule-based systems. By leveraging the medical room sensor data to automatically identify the process type, the method can provide more targeted insights and recommendations for process optimization, ultimately leading to enhanced operational efficiency and quality of care. Including the determination of the process indicator enhances the accuracy, relevance, and effectiveness of the method in assessing and improving the operational performance of processes in the medical facility.

Next the determined 1200 process parameter is associated 1300 with at least one process indicator. A process indicator represents a specific process type taking place in the medical room. For example, the process type could be surgical procedures, patient monitoring, or laboratory tests. The association links the process parameter to the specific process type.

The method 1000 retrieves 1400 at least one historical process parameter associated with the process indicator. This historical data 1400 can represent previous measurements 1410 or benchmarks 1420 related to the same process type in the medical room. The historical data can provide a reference point for evaluating the current operational performance, as is shown in FIG. 2.

The determined process parameter is normalized 1500 based on the provided historical process parameter. Normalization 1500 ensures that the current measurement is compared and adjusted against the historical data to account for any variations or changes over time. This step allows for a standardized and comparable assessment of the operational performance in the context of the process occurring. Finally, the method outputs 1600 the normalized process parameter, which represents the operational performance of the process in the medical room. This output can be utilized for analysis, visualization, reporting, or further decision-making processes related to optimizing the performance of the process. Based on the above it will be clear that the advantage of the method 1000 is based on the insight that by implementing the computer-implemented method 1000, medical facilities can effectively monitor and evaluate the operational performance of processes within their medical rooms. The method 1000 enables the identification of process parameters, association with process indicators, historical benchmarking, normalization, and outputting of normalized process parameters for improved process management and performance optimization.

The following exemplary embodiments, although not illustrated in any figure, embody the principles of the present invention and are included within its scope.

According to a first exemplary embodiment the process parameter is indicative for the ergonomic position of at least one person in the medical room. An ergonomic position refers to the posture or body alignment of a person that minimizes strain, discomfort, and the risk of musculoskeletal injuries during work or activities. It involves positioning the body in a way that promotes comfort, efficiency, and reduces the risk of repetitive strain or overexertion. In a healthcare setting, maintaining an ergonomic position is crucial for both healthcare professionals and patients. It is important to note that specific ergonomic considerations may vary depending on the specific healthcare task or procedure being performed, as well as individual factors such as body size, mobility, and any existing medical conditions. An ergonomic position can for example be neutral spine alignment, proper body mechanics, supportive seating, work surface height, proper use of equipment, and/or regular breaks and movement. Maintaining a neutral spine alignment helps to distribute the body's weight evenly and reduce stress on the spine. It involves keeping the natural curves of the spine intact, with the head, neck, and back aligned in a neutral position. When the determined process parameter is indicative for the ergonomic position, it can also aid in assessing whether the person in the medical room performs proper body mechanics to in their tasks. This includes techniques for lifting, transferring patients, and using equipment in a way that minimizes strain on the body. Moreover, ensuring that work surfaces, such as desks or examination tables, are at an appropriate height can help maintain a neutral posture and prevent unnecessary strain on the neck, back, or upper extremities. By assessing ergonomic positions, the method 100 can reduce the risk of musculoskeletal injuries and work-related discomfort with healthcare professionals. Additionally, patients may benefit from ergonomic positioning during medical procedures or when using assistive devices to ensure their comfort and well-being. Furthermore, it is more preferable for the method 1000 to include the step of estimating the pose of the at least one person in the medical room during the determining of the at least one process parameter. Estimating the pose involves analysing the video data or sensor data to determine the spatial configuration and position of the person's body parts. This can be achieved using techniques such as pose estimation, skeleton tracking, or motion capture. By estimating the pose of the person, the method gains the ability to assess the ergonomic position and alignment of a body of a person in the medical room. The process parameter derived from this assessment indicates whether the person's posture is ergonomically optimal or whether there are potential issues that could lead to discomfort, fatigue, or injury. Including ergonomic considerations and pose estimation in the computer-implemented method allows for a more comprehensive evaluation of the operational performance in the medical room. It helps identify potential ergonomic risks, evaluate the impact of body positioning on efficiency and safety, and provide feedback or recommendations to improve the ergonomic conditions for the personnel involved in the processes. By integrating ergonomic considerations and pose estimation, the method contributes to creating a healthier and safer work environment in the medical facility, ultimately enhancing the well-being and productivity of the personnel while optimizing the overall operational performance.

In order to analyse and monitor changes in the pose of the person over time, it is preferred that the computer-implemented method 1000 further comprises tracking of variations of the pose estimation between digital images comprised in the video data. In this way insights into their movement patterns and potential ergonomic issues are provided. By tracking variations of the pose estimation, the method 1000 can detect and quantify any deviations or fluctuations in the person's posture and body position. This information can be used to identify repetitive or prolonged movements, postural instability, or other factors that may contribute to ergonomic challenges or potential musculoskeletal issues. The tracking of pose estimation variations adds a dynamic aspect to the evaluation of ergonomic positions in the medical room. It enables the method 1000 to capture not only static snapshots of the person's posture but also the changes and dynamics of their movements throughout the video data. This temporal analysis enhances the understanding of ergonomic risks and helps in identifying trends or patterns that may require intervention or adjustment. By incorporating the tracking of variations in pose estimation, the computer-implemented method 1000 provides a more comprehensive and detailed assessment of the ergonomic conditions and movement behaviours in the medical room. This information can be used to guide interventions, optimize workflow, and enhance the overall ergonomic design and practices in the medical facility. In this exemplary embodiment it will be clear that a sequences of frames 1130 and/or a multi-sourced sequences of frames 1140 is required.

In order to evaluate the alignment and positioning of the estimated pose in relation to a predetermined standard or reference pose, which represents the ergonomic baseline, the step of determining the ergonomic process parameter can comprise comparing the estimated pose to a baseline, and calculating at least one pose score based on the comparison. Furthermore, it is more preferable for the method to include the step of outputting the calculated pose score as the ergonomic process parameter. The pose score represents a quantitative measure of how closely the estimated pose aligns with the ergonomic baseline. It provides a numerical indication of the ergonomic quality of the person's posture and serves as a metric to assess their ergonomic performance. Additionally, it is preferable to include the step of comparing the calculated pose score to historical process data that contains a process indicator indicative of the same process type in the medical room. By comparing the pose score to historical process data, the method establishes a context-specific evaluation of the ergonomic performance. This comparison allows for benchmarking the current pose score against past measurements or benchmarks associated with the same process type, enabling insights into the progress or deviations over time. By incorporating the steps of comparing the estimated pose to a baseline, calculating pose scores, outputting the pose scores as the ergonomic process parameter, and comparing the pose scores to historical process data, the computer-implemented method 1000 provides a comprehensive framework for assessing and monitoring ergonomic performance. This approach enables the identification of potential ergonomic issues, the evaluation of improvements or deteriorations, and the establishment of evidence-based guidelines for optimal ergonomic practices in the medical facility.

Preferably, the method 1000 further comprises outputting one or more pose proposals based on the comparison, wherein the one or more pose proposals represent at least part of a pose improvement to be carried out on a corresponding real body part of the at least one person in the medical room such that a difference between the calculated at least one pose score and the historical process data is reduced. These pose proposals represent suggested improvements to be made to the corresponding real body part of the person in the medical room. The aim of these proposals is to reduce the difference between the calculated pose score and the historical process data, indicating a closer alignment with optimal ergonomic conditions. It is noteworthy that the optimal ergonomic condition can be contextually determined based on the process type. For example, an upright ergonomic position of a person in the medical room may be the best-in-class without any process going on. However, when a surgical procedure is occurring, it is possible that the upright position is not possible due to the surgical steps needing to be performed. The optimal ergonomic position can therefore be process dependent. Furthermore, it is more preferable to use RULA, Rapid Upper Limb Assessment, and/or REBA, Rapid Entire Body Assessment, scoring methods in the calculation of the pose scores. RULA and REBA are ergonomic assessment tools that provide structured methodologies for evaluating and scoring the ergonomic risks associated with body postures and movements. These scoring systems assign scores based on various factors such as posture, force exertion, repetition, and duration. By incorporating RULA and/or REBA scoring in the calculation of the pose scores, the method applies recognized and validated ergonomic assessment methods. This allows for a standardized and objective evaluation of the ergonomic quality of the estimated pose. The pose proposals generated based on these scores provide actionable recommendations for improving the ergonomics of the person's posture and movements. The outputted pose proposals guide the person in making adjustments to their body position, posture, or movements to achieve a more optimal ergonomic alignment. By implementing these suggested improvements, the difference between the calculated pose score and the historical process data is expected to decrease, indicating progress towards better ergonomic performance. In summary, the inclusion of the step to output pose proposals, along with the use of RULA and/or REBA scoring, enhances the practical application of the computer-implemented method. It provides actionable recommendations for improving ergonomic conditions, promotes healthier and safer work practices, and facilitates the reduction of discrepancies between calculated pose scores and historical process data.

According to a further exemplary embodiment, the process parameter can be indicative for a first difference between a first time stamp at which a first process type takes place and a second time stamp at which a second process type takes place. The first process type can corresponds to a first incision and the second process type can correspond to a wound closure of at least one person in the medical room. In other words, such a process parameter indicates the time difference between the occurrence of two specific events in the medical room: the first process type, which involves making for example a deliberate incision, and the second process type, which involves the closure of the wound. The time stamp refers to a recorded data point that marks the exact time when an event or action occurred. It serves as a reference for tracking, analysis, and synchronization purposes. For the sake of completeness it is noted that an incision refers to a controlled surgical cut made in the body to gain access to a specific area for medical interventions. Surgeons and medical practitioners perform incisions using specialized instruments like scalpels, surgical scissors, or lasers. Wound closure, on the other hand, involves bringing together and securing the edges of the incision to facilitate healing and minimize the risk of infection. Wound closure involves bringing the edges of the incision together and securing them to promote healing and minimize the risk of infection. A process parameter which represents the difference between the first time stamp when the first incision is made, and the second time stamp when the incision is closed, is indicative for the efficiency of the surgical process and has secondary effects on the treatment quality. For instance a shorter process parameter which represents the difference between the first time stamp and the second time stamp results in shorter sedation periods which leads to better recovery and reduction of other risks.

Other time process parameters can also be determined, for example, a parameter indicative for a timely start of the day. Such a time parameter focuses on the planned start time of the first surgery. It examines how often the surgical team begins the day's operations at the scheduled time and measures any deviations from the planned start time, whether it occurs earlier or later. Further time parameters include a first patient on time parameter. This parameter evaluates whether the first surgery starts as scheduled. It assesses whether the surgical procedure begins at the planned time or if there are delays. Furthermore, a next patient on time/team waiting for next surgery time parameter can be determined. This parameter looks at the time intervals between surgeries. It examines if each surgery starts according to the predetermined schedule and measures any waiting or idle time between procedures. Such a time parameters helps identify if the time between surgeries is consistent with the planned schedule or if there are delays impacting patient flow. Furthermore, a draping time parameter can be determined. This parameter measures the time spent on patient draping, which involves preparing the patient and the surgical site for the procedure. It helps assess the efficiency of the draping process and identifies any potential areas for improvement in time management. Further time parameters can include an idle time or a cleaning time. The idle time parameter calculates the total minutes during which the entire surgical team or individual team members are unable to work on productive tasks. It highlights periods of inactivity or waiting, which can be further analyzed to optimize workflow and reduce unnecessary downtime. The cleaning time parameter focuses on the time spent on cleaning activities between surgeries. It includes tasks such as sterilization, equipment preparation, and room cleaning. Monitoring cleaning time helps ensure proper infection control measures and allows for efficient turnaround between procedures. By tracking and analyzing these time parameters, healthcare facilities can gain insights into their operational efficiency, identify bottlenecks, optimize scheduling, minimize delays, and enhance overall patient care and satisfaction. These parameters provide valuable data for process optimization, resource allocation, and quality improvement initiatives within the hospital environment. It will be clear that one or more time process parameters can be determined. It will be clear that the time process parameters can be obtained in combination with the ergonomic process parameters and vice versa.

The step of normalizing can be process parameter dependents. For example, when a time process parameter is used, the normalizing can be performed differently than the process parameter. According to an exemplary embodiment the step of normalizing 1500 the determined ime process parameter further comprises comparing the first difference to historical process data containing a process indicator which is indicative for the same process type. This normalization step 1500 allows for a contextual assessment of the change in the process parameter in relation to past measurements or benchmarks associated with the same process type. By comparing the first difference to historical process data, the method 1000 establishes a reference point to evaluate the significance and impact of the change in the process parameter. In this way the method provides a means to assess whether the observed change is within expected ranges or if it deviates from historical patterns. The comparison to historical process data allows for the identification of trends, anomalies, or potential issues in the operational performance of the process. It helps determine whether the change in the process parameter is consistent with past behaviour or if it requires further investigation or intervention. By incorporating this normalization step 1500, the computer-implemented method 10000 ensures that the assessment of the process parameter takes into account the historical context and process-specific considerations and provides a more accurate and meaningful evaluation of the operational performance, enabling timely identification of deviations and the implementation of appropriate measures to address them. In summary, the normalization step 1500 which involves comparing the first difference to historical process data, enhances the method's 1000 ability to analyse and interpret the changes in the determined process parameter. It contributes to a more comprehensive understanding of the process performance and supports informed decision-making for process optimization and improvement. The difference can be associated with historical differences measured for example for the same team in the same setting, or in another setting, or another team. It is further preferred that the historical process data containing the process indicator which is indicative for the same process type comprises a minimal historical difference between a first time stamp at which a first process type takes place and a second time stamp at which a second process type takes place, and a maximum historical difference between a first time stamp at which a first process type takes place and a second time stamp at which a second process type takes place, wherein the normalizing comprises performing a ration between a difference between the first difference and the minimal difference to a difference between the maximum difference and the minimum difference. The observed difference can be normalised by combining the difference with a large set of historical measurements in different settings, for instance by comparing to the shortest and longest documented difference of a group or a subgroup, e.g. all hospitals globally, all private clinics, all teams of the same medical department etc.

Moreover, other process parameters can be determined using the method according to further exemplary embodiment the process parameters can also be indicative of various aspects related to the physical setup and usage of resources in the operating room. For example a process parameter can be indicative of an amount of instruments brought into the operating room. This parameter measures the number of instruments and equipment brought into the operating room for a specific procedure. It helps assess the efficiency of instrument preparation and ensures that an adequate supply of instruments is available for the surgical team. A further process parameter can be indicative of a number of tables, for example instrument tables. This parameter focuses on the number of tables used in the operating room for organizing and storing instruments. It helps evaluate the optimal allocation of tables and ensures that sufficient space is available for instrument setup and easy accessibility during the procedure. Moreover, process parameter can be indicative of a use of specific devices, for example a C-arm, a canister, etc. Such a parameter tracks the usage of specific devices during surgical procedures. For example, it monitors the utilization of a C-arm for intraoperative imaging or the frequency of canister usage for waste disposal. This information assists in resource planning, maintenance, and evaluating the efficiency of device utilization. Further process parameters can be indicative of a placement of instrument table. This parameter examines the positioning of the instrument table within the operating room. It assesses whether the table is optimally placed for easy access by the surgical team, minimizing unnecessary movements and enhancing workflow efficiency. Other process parameter can be indicative of a tray setup (layout) on the table standardization, an open and close time, a percentage of room used per day. A tray setup, for example the layout, on the table standardization parameter focuses on the consistency and standardization of tray setups on the instrument table. It ensures that the layout of instruments and equipment is standardized and organized in a manner that promotes efficiency, reduces errors, and facilitates the smooth progression of the surgical procedure. An open and close time parameter measures the time taken to open and close the operating room for a specific procedure. It helps evaluate the efficiency of room setup and turnover, ensuring timely readiness for subsequent surgeries and optimizing resource utilization. A parameter indicative of a percentage of room used per day calculates the percentage of time that the operating room is occupied and actively used for surgical procedures during a given day. It provides insights into room utilization and can aid in optimizing scheduling, maximizing efficiency, and minimizing idle time. Monitoring and analysing these process parameters related to physical setup and resource usage in the operating room can lead to improvements in efficiency, cost-effectiveness, and patient care. It allows for the identification of areas where standardization, organization, and utilization can be enhanced to optimize surgical processes and resource allocation.

Physical activities can play a crucial role as process parameters and indicators in healthcare settings. It is therefore advantageous for the method to determined a process parameter indicative for preparing instruments as the related indicators can provide valuable insights into efficiency and workflow. For example, assessing the number of trays used for instrument preparation aids in evaluating the adequacy of the instrument supply and ensures that the required instruments are available for the surgical team in a timely manner. Further, a parameter indicative for a positioning of instrument trays in the room focuses on how the instrument trays are positioned within the room. It examines whether the trays are strategically placed to facilitate easy access and efficient workflow during the surgical procedure. a parameter indicative for a instrument transportation evaluates the methods used to transport instrument trays into the operating room. It compares whether the trays are manually carried one by one or wheeled in using a cart. Assessing the transportation method provides insights into the efficiency and ergonomics of instrument delivery, potentially identifying opportunities for improvement. By monitoring and analysing these indicators, healthcare facilities can identify areas for process optimization. For example, implementing a more efficient method of instrument transportation, such as using carts, can save time and reduce physical strain on staff. Ensuring proper positioning of instrument trays can enhance accessibility and minimize unnecessary movements during procedures. These improvements can contribute to streamlining workflows, reducing errors, and enhancing overall efficiency in instrument preparation processes.

According to yet another exemplary embodiment, the NASA Task Load Index (TLX) can be a valuable process parameter for assessing the cognitive workload experienced by healthcare professionals during surgeries. Normalizing the TLX across different surgery types comprises dividing the average TLX of each surgery type by the highest TLX allows for a comparative analysis of workload variations within the same team. This normalization method 1500 provides insights into the relative differences in workload across surgery types performed by the same team. By utilizing normalized TLX values, healthcare facilities can strategically plan and optimize surgical schedules based on task load considerations. For example, alternating surgeries with significantly higher TLX values with those having lower TLX values can help balance the workload and prevent excessive strain on the surgical team. Similarly, scheduling surgeries with higher TLX values during the morning and lower TLX values during the afternoon can help manage cognitive fatigue and maintain optimal performance. Noise levels in operating rooms can impact the working environment and potentially affect the concentration and communication of the surgical team. Measuring and monitoring noise levels in surgery rooms is important for creating a conducive and safe environment. Normalizing noise levels with other surgery types, hospitals, or established standards can provide benchmarks for evaluating and improving the acoustic conditions within operating rooms. This normalization allows for comparisons and identification of potential issues or areas requiring noise reduction interventions.

By incorporating normalized TLX and noise level parameters into surgical planning and facility management processes, healthcare facilities can enhance efficiency, team well-being, and patient safety.

In yet another exemplary embodiment, the process parameter is indicative for at least one of an amount of verbally communicated words, a pace of oral communication, an amount of simultaneous oral communications, a time period between two oral communications. Put differently, it is preferable for the process parameter to be indicative of various aspects of oral communication. These aspects can include an amount of verbally communicated words such a process parameter can quantify the number of words spoken during a given period. It provides insights into the volume of information exchanged through oral communication. Another aspect of oral communication can be the pace of oral communication. Such a process parameter can measure the speed or rate at which oral communication occurs. It indicates the tempo or cadence of spoken interactions. Another aspect of oral communication can be the amount of simultaneous oral communications. This process parameter can assess the number of concurrent oral communications happening within a specific context or environment. It helps evaluate the level of multitasking or overlapping conversations. Yet another aspect of oral communication can be the time period between two oral communications. this process parameter can determine the duration between successive oral communications. It provides information about the intervals or gaps between spoken interactions. By utilizing these process parameters, the computer-implemented method can analyse and monitor various aspects of oral communication in a medical facility or any relevant context. These parameters offer insights into communication patterns, efficiency, and potential challenges within the oral communication process. The method can capture, measure, and analyse these process parameters using appropriate sensor data, such as audio recordings, speech recognition technologies, or other relevant sources. This enables a quantitative evaluation of oral communication performance and facilitates targeted improvements to enhance communication effectiveness, efficiency, and patient care in the healthcare facility. In summary, the inclusion of process parameters related to the amount of verbally communicated words, pace of oral communication, amount of simultaneous oral communications, and time period between two oral communications enhances the method's ability to evaluate and optimize oral communication practices in the medical facility or other relevant settings.

FIG. 2 illustrates that the sensor data can be received 1100 from various sources, such as camera's, manual observations, data logging systems, or other monitoring and measurement techniques. The sensor data can comprises audio data which captures sound information, which can be valuable for assessing noise levels, communication quality, or the presence of specific sounds relevant to the process being evaluated. The sensor data can be comprises of a single frame 1110, multi-sourced single frames 1120, a sequences of frames 1130 and/or a multi-sourced sequences of frames 1140. A single frame 1110 refers to a static image captured by a sensor, such as a camera, at a specific moment in time. It can provide visual information about the patient, medical equipment, or the environment within the medical room. Single frames can be useful for analysing static conditions or objects. Multi-sourced single frames 1120 involve the use of multiple sensors capturing individual frames simultaneously. These sensors may be positioned at different angles or locations, providing different perspectives of the same scene. By combining these multiple frames 1120, a more comprehensive and detailed understanding of the medical room or the patient's condition can be obtained.

Sensor data can also be in the form of a sequence of frames 1130, captured over a period of time. This can be obtained through video recordings or continuous monitoring using sensors such as cameras. Sequences of frames 1130 enable the observation of dynamic processes, movements, or changes in the medical room or the patient's condition. A sequences of frames 1130 can also be called video data which provides visual information about the medical room, including the layout, equipment, and activities taking place. Similar to multi-sourced single frames 1120, a multi-source sequence of frames 1140 involves the use of multiple sensors capturing continuous frames over time. These sensors may have different viewpoints or perspectives, allowing for a more comprehensive understanding of the evolving situation or process being monitored. The use of sensor data in different formats and configurations allows for a more holistic and detailed analysis of medical processes. It enables the method to observe, measure, and analyse various aspects of the environment, patient interactions, or medical procedures to enhance patient care, optimize workflow, and improve operational efficiency in the hospital setting. In a preferred embodiment, said medical room sensor data comprises video data of the medical room, and/or audio data obtained in the medical room. It is noted that audio data is not illustrated in FIG. 2 but can be simultaneously obtained using a video camera for example. More preferably, said medical room sensor data comprises multi-source medical room sensor data 1120, 1140, wherein at least two of the sources comprises a respective intake angle of the medical room. In the computer-implemented method 1000, it is preferable to include video data and/or audio data of the medical room as part of the medical room sensor data. Additionally, it is more preferable to include multi-source medical room sensor data 1120, 1140, where at least two of the sources provide a respective intake angle of the medical room. The intake angle refers to the perspective or viewpoint from which the medical room is observed or captured. By incorporating multiple intake angles, the method gains a more comprehensive and nuanced understanding of the medical room's environment, activities, and process performance. By including video data, audio data, and multi-source sensor data with various intake angles, the computer-implemented method 1000 can leverage a rich and diverse set of information for determining process parameters and assessing operational performance. The combination of visual and audio cues, along with multiple perspectives, provides a more holistic view of the medical room and its processes. This approach enhances the accuracy and effectiveness of the method in analysing and evaluating the operational performance of processes. It allows for a deeper understanding of the context, interactions, and potential influencing factors within the medical room, contributing to more informed decision-making and optimization efforts.

In yet another exemplary embodiment, several parameters related to instruments can be used and normalized to assess efficiency and optimize instrument utilization in healthcare settings. For instance, a process parameter indicative for a number of instruments used measures the total number of instruments used during a specific procedure. It provides insights into the instrument requirements for different surgeries and helps optimize inventory management. In another instance, a process parameter indicative for a number of instruments versus number of trays compares the number of instruments used with the number of trays utilized for instrument storage and organization. It helps evaluate the efficiency of tray utilization and identifies potential discrepancies between instrument needs and tray capacity. In another instance, a process parameter indicative for a percentage of instruments opened but not used calculates the percentage of instruments that are opened in the operating room but remain unused during the procedure. It provides insights into instrument waste and highlights opportunities for optimizing instrument selection and utilization. In another instance, a process parameter indicative for a percentage of single-use instruments determines the percentage of instruments designated for single-use in a surgical procedure. It ensures compliance with infection control protocols and helps monitor the appropriate utilization of disposable instruments. By normalizing 1500 these parameters, healthcare facilities can compare their instrument utilization against benchmarks from other hospitals or different surgery types. This normalization 1500 enables the identification of best practices, areas for improvement, and potential cost-saving opportunities. It also facilitates data-driven decision-making, resource optimization, and standardization across healthcare settings.

Maintaining sterility is of utmost importance in healthcare settings. Various process parameters can be used as indicators to monitor and optimize sterility practices. Herebelow several exemplary embodiments of process parameters related to sterility maintenance are described. For instance, a process parameter indicative for a number of times doors are Opened measures the frequency of door openings during surgical procedures. It helps evaluate the adherence to sterile protocols and identifies opportunities to minimize door openings, reducing the risk of contaminants entering the operating room. a process parameter indicative for a duration of door openings measures the length of time the doors remain open during surgical procedures and provides insights into the extent of potential exposure to non-sterile environments and helps identify opportunities to minimize door opening durations, thereby reducing the risk of sterility compromise. In yet another instance, a process parameter indicative for a Time Instruments Remain Opened until Use measures the duration between opening sterile instrument packs and their actual use in the surgical procedure and evaluates efficiency in instrument handling and ensures that instruments are used promptly to maintain sterility. By normalizing 1500 these parameters, hospitals can compare the performance of different surgery rooms with varying entry layouts. For example, operating rooms with a separate induction room or a separate room for instrument preparation may have different door-related indicators due to their specific layout and workflow. Normalizing these indicators based on the specific room layout allows for fair comparisons and benchmarking against best practices within similar environments. By monitoring and optimizing these sterility-related parameters, healthcare facilities can enhance infection control practices, minimize the risk of surgical site infections, and promote patient safety. It also aids in the development of standardized protocols for maintaining sterility across different surgical settings.

In yet another exemplary embodiment walking lines within the medical room are mapped to provide valuable insights into movement patterns and help assess various aspects of OR efficiency and sterility maintenance. In this context walking lines are a process parameter, and mapping walking lines can be seen as determining a process parameter. According to a first exemplary embodiment ai-based person detection and movement tracking is used to map the walking lines. By utilizing AI algorithms for person detection and movement tracking on video footage captured within the medical room, it becomes possible to map the walking lines of healthcare professionals. This technology can identify and track the movement of individuals, allowing for the extraction of data such as distances walked and patterns of movement. The AI algorithms as such, is known and commercially available and will not be elaborated for the sake of simplicity. When mapping walking lines from different medical rooms, it may be necessary to align the extremes of these lines. By aligning the walking lines, it becomes easier to compare movement intensity, identify high-traffic areas near the surgical table, and assess efficiency-related factors. This alignment can be achieved by expanding or contracting the walking lines to achieve consistency. The alignment By comparing walking lines from different medical rooms, it becomes possible to analyze and compare movement patterns, identify areas of potential improvement, and assess the impact of different factors on movement efficiency. This analysis can be done without detailed medical room data, as long as the cameras are mounted in similar positions. Combining walking line data with timestamps allows for a focus on specific phases of the surgical process. Additionally, if the video includes indications of the sterility area, such as laminar flow, the walking lines can help identify potential risks to sterility. By combining walking line data with time information, it becomes possible to determine if and when sterility breaches occur and identify critical moments, such as those before wound closure. By leveraging AI tools to map walking lines and combining this data with timestamps and indications of sterility, healthcare facilities can gain valuable insights into OR efficiency, movement patterns, and sterility maintenance. This information can support process optimization, infection control efforts, and the implementation of best practices in surgical settings. Considering the environmental impact of healthcare facilities is crucial for sustainable practices. A process parameter such as the number of waste bins and the weight of waste can provide insights into the amount of waste generated. It can be further categorized into medical waste, non-risk waste, and recyclable waste to assess the efficiency of waste management practices. Monitoring the total sterilization of instruments (trays opened) and the amount of consumables used can help evaluate resource utilization and identify opportunities for waste reduction. Additionally, tracking the use of single-use instruments and consumables can provide data on the impact of disposable items. Measuring water consumption in sterilization processes can highlight areas where water efficiency improvements can be made. Identifying water usage patterns and optimizing sterilization practices can contribute to reducing water consumption. Tracking consumables that were opened but not used can indicate inefficiencies in inventory management and potentially lead to unnecessary waste. Monitoring and minimizing the use of unused consumables can reduce environmental impact. Broad indicators such as energy spent on heating, cooling, and ventilation systems can provide insights into the energy efficiency of the facility. Monitoring energy consumption and implementing energy-saving measures can contribute to reducing the environmental footprint. By considering these process parameters and indicators, healthcare facilities can assess their environmental impact, identify areas for improvement, and implement sustainable practices to minimize waste generation, water consumption, and energy usage. This not only reduces the facility's ecological footprint but also promotes responsible and sustainable healthcare practices.

FIG. 2 further illustrates that, in exemplary embodiment, the historical process data 1400 containing the process indicator which is indicative for the same process type comprises previously determined process parameters 1410 associated with the process indicator indicative for the same process type and/or predetermined process parameters 1420 associated with the process indicator indicative for the same process type, wherein the predetermined process parameters 1410 represent best practice process parameters for said process type. Firstly, previously determined process parameter parameters are associated with the process indicator indicative of the same process type. The previously determined process parameters 1410 represent measurements or benchmarks that have been previously determined or recorded for similar processes in the medical room, for example an knee replacement performed by the same medical team. This data provides a historical reference to assess the current operational performance. Alternatively or additionally, the historical process data can also include predetermined process parameters 1420 associated with the process indicator indicative of the same process type. These predetermined process parameters 1420 represent the best practice or ideal values for the specific process type in the medical room. They serve as a standard or target to evaluate and optimize the operational performance. By incorporating both previously determined process parameters and predetermined process parameters, the method enables a comprehensive evaluation of the current operational performance. The previously determined historical data provides insights into past performance, while the predetermined process parameters represent an optimal or desired state for the process type. This combination allows for a more robust assessment and facilitates the identification of areas for improvement or adherence to best practices.

FIG. 2 further illustrates that the computer-implemented method 1000 can further comprise receiving 1150 medical room process data containing at least one process indicator which is indicative for a process type in said medical room. More preferably, the computer-implemented method 1000 further comprises the steps of including 1610 the determined process parameter associated with the process indicator in the historical process data 1400. In the computer-implemented method, it is preferable to include the step of receiving medical room process data containing at least one process indicator that is indicative of a process type in the medical room. This additional step allows for the incorporation of process-specific information and measurements into the analysis and evaluation of the operational performance. Furthermore, it is more preferable to include the step of including the determined process parameter associated with the process indicator in the historical process data. This ensures that the newly determined process parameter is added to the existing historical data for that particular process type. By including the determined process parameter, the historical process data becomes more comprehensive and up-to-date, facilitating a more accurate assessment and comparison of the operational performance over time. By receiving medical room process data and including the determined process parameter sin the historical process data, the computer-implemented method enables a continuous and dynamic monitoring and evaluation of the operational performance of processes in the medical room. The method can adapt to changes and updates in the process parameters, ensuring that the assessment remains relevant and reflective of the current state of the process. This improved approach allows for a more informed analysis, benchmarking, and optimization of the operational performance.

Whilst the principles of the present invention have been set out above in connection with specific embodiments, it is to be understood that this description is merely made by way of example and not as a limitation of the scope of protection which is determined by the appended claims.

1. A computer-implemented method for defining at least one process parameter being indicative of an operational performance of a process in a medical facility, the method comprising the steps of:
receiving medical room sensor data monitored in said at least one medical room of said medical facility;
determining, from said received medical room sensor data, at least one process parameter which is indicative for the operational performance of the process in said medical room;
associating at least one process indicator with the determined process parameter, wherein a process indicator is indicative for a process type in said medical room;
providing at least one historical process parameter associated with the process indicator;
normalizing said determined process parameter on the basis of the provided at least one historical process parameter; and
outputting the normalized process parameter.

2. The computer-implemented method according to claim 1, wherein the historical process data containing the process indicator which indicative for the same process type comprises previously determined process parameters associated with the process indicator indicative for the same process type and/or predetermined process parameters associated with the process indicator indicative for the same process type, wherein the predetermined process parameters represent best practice process parameters for said process type.

3. The computer-implemented method according to claim 1, further comprising determining the process indicator from the medical room sensor data.

4. The computer-implemented method according to claim 1, further comprising receiving medical room process data containing at least one process indicator which is indicative for a process type in said medical room.

5. The computer-implemented method according to claim 4, further comprising the steps of including the determined process parameter associated with the process indicator in the historical process data.

6. The computer-implemented method according to claim 1, wherein said medical room sensor data comprises video data of the medical room, and/or audio data obtained in the medical room.

7. The computer-implemented method according to claim 6, wherein said medical room sensor data comprises multi-source medical room sensor data, wherein at least two of the sources comprises a respective intake angle of the medical room.

8. The computer-implemented method according to claim 1, wherein the process parameter is indicative for the ergonomic position of at least one person in the medical room.

9. The computer-implemented method according to claim 8, further comprising estimating a pose of the at least one person in the medical room, wherein the process parameter is indicative for the ergonomic position of the pose of the person in the medical room.

10. The computer-implemented method according to claim 9, wherein the historical process data containing the process indicator which indicative for the same process type comprises previously determined process parameters associated with the process indicator indicative for the same process type and/or predetermined process parameters associated with the process indicator indicative for the same process type, wherein the predetermined process parameters represent best practice process parameters for said process type; and further comprising tracking of variations of the pose estimation between digital images comprised in the video data.

11. The computer-implemented method according to claim 5, wherein the step of determining the ergonomic process parameter comprises comparing the estimated pose to a baseline; and calculating at least one pose score based on the comparison.

12. The computer-implemented method according to claim 11, outputting the calculated at least one pose score as the ergonomic process parameter.

13. The computer-implemented method according to claim 7, further comprising comparing the calculated at least one pose score to historical process data containing a process indicator which is indicative for the same process type in said medical room.

14. The computer-implemented method according to claim 9, outputting one or more pose proposals based on the comparison, wherein the one or more pose proposals represent at least part of a pose improvement to be carried out on a corresponding real body part of the at least one person in the medical room such that a difference between the calculated at least one pose score and the historical process data is reduced.

15. The computer-implemented method according to claim 7, wherein the step of calculating the at least one pose score comprises a RULA/REBA scoring.

16. The computer-implemented method according to claim 1, wherein the process parameter is indicative for a first difference between a first time stamp at which a first process type takes place and a second time stamp at which a second process type takes place, wherein the first process type corresponds to a first incision and the second process type corresponds to a wound closure of at least one person in the medical room.

17. The computer-implemented method according to claim 16, wherein the step of normalizing further comprises comparing the first difference to historical process data containing a process indicator which is indicative for the same process type. The difference can be associated with historical differences measured for example for the same team in the same setting, or in another setting, or another team.

18. The computer-implemented method according to claim 17, wherein the historical process data containing the process indicator which is indicative for the same process type comprises a minimal historical difference between a first time stamp at which a first process type takes place and a second time stamp at which a second process type takes place, and a maximum historical difference between a first time stamp at which a first process type takes place and a second time stamp at which a second process type takes place, wherein the normalizing comprises performing a ration between a difference between the first difference and the minimal difference to a difference between the maximum difference and the minimum difference. The observed difference can be normalised by combining the difference with a large set of historical measurements in different settings, for instance by comparing to the shortest and longest documented difference of a group or a subgroup, e.g. all hospitals globally, all private clinics, all teams of the same medical department etc.

19. The computer-implemented method according to claim 1, wherein the process parameter is indicative for at least one of an amount of verbally communicated words, a pace of oral communication, an amount of simultaneous oral communications, a time period between two oral communications.

20. The computer-implemented method according to claim 1, wherein the process parameter is indicative for a movement path for one or more objects in the medical room, and wherein the normalizing comprises comparing the movement path to historical process data containing a process indicator which is indicative for the same process type.

21. The computer-implemented method according to claim 20, wherein the normalizing further comprises overlaying the historical movement path data on to the determined movement path.

22. The computer-implemented method according to claim 2, further comprising indexing the video data

23. A computer program product comprising a computer-executable program of instructions for performing, when executed on a computer, the steps of the method of claim 1.
